(19) United States
(12) Patent Application Publication
(10) Pub. No.: US 2007/0238905 A1
(43) Pub. Date: Oct. 11, 2007

(54) Processes for converting glycerol to glycerol ethers

(76) Inventors: Victor Manuel Arredondo, West Chester, OH (US); Deborah Jean Back, Cleves, OH (US); Patrick Joseph Corrigan, Glendale, OH (US); David Patrick Kreuzer, Fairfield, OH (US); Angella Christine Cearley, Hamilton, OH (US)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(21) Appl. No.: 11/731,616

(22) Filed: Mar. 30, 2007

Related U.S. Application Data
(60) Provisional application No. 60/789,618, filed on Apr. 5, 2006.

(51) Int. Cl. C07C 41/09

(52) U.S. Cl. 568/672

(57) ABSTRACT

Processes for converting glycerol to alkyl glycerol ethers that involve providing glycerol, alkyl alcohol and an etherification catalyst, providing an etherification reactor and, adding the glycerol, alkyl alcohol and etherification catalyst to the etherification reactor and reacting the glycerol, alkyl alcohol and etherification catalyst in the etherification reactor to obtain a reaction product comprising alkyl glycerol ethers.

### CROSS REFERENCE TO RELATED APPLICATIONS

This application claims priority under 35 U.S.C. § 119(e) to U.S. Provisional Application Ser. No. 60/789,618 filed Apr. 5, 2006.

### FIELD OF THE INVENTION

Embodiments of the present invention generally relate to processes for converting glycerol to glycerol ethers, and in particular, converting crude glycerol to alkyl glycerol ethers.

### BACKGROUND OF THE INVENTION

Alkyl glycerol ethers are ether lipids that may be used in a variety of applications as antimicrobials, emulsifiers, surfactants, fragrance enhancers, moisture retaining agents, solvents, solvatropes and the like. Appending one, two or three alkyl chain hydrophobes and/or capping groups onto glycerol allows alkyl glycerol ethers to possess unique physico-chemical and solubility properties that allow alkyl glycerol ethers to be tailored for multiple uses as set forth previously.

As described in J. Am. Oil Chem. Soc., 65:1299 (1988), one conventional method for producing alkyl glycerol ethers involves hydrolyzing alkyl glycidyl ethers synthesized from an alcohol and epichlorohydrin under alkaline conditions. One problem with this process is that it is stoichiometric and, therefore, can generate large quantities of undesired waste and higher organochloride byproducts. Additionally, this process includes the use epichlorohydrin, which is toxic and can be difficult to handle.

Another conventional process used to prepare alkyl glycerol ethers involves Williamson's ether synthesis. Using this process, polyols can be reacted with alkyl halides or alkali metal salts of alkyl sulfates in the presence of strong bases. However, similar to the previously described process, Williamson's ether synthesis is also stoichiometric, and thus, can produce large quantities of undesired salts and waste. Additionally, this process is unselective since either the primary or secondary hydroxyl group can react. This inability to be selective can generate mono-, di-, and tri-alkylated products and byproducts, which, in turn, can make production costs high as undesired byproducts may need to be removed prior to further use.

Yet another conventional method for producing alkyl glycerol ethers involves reacting a carbonyl compound, such as an aldehyde and/or a ketone, with glycerol in the presence of an acid catalyst and hydrogenating the resulting acetal and/or ketal in the presence of a supported or unsupported palladium catalyst. See, for example, EP0624563. This method has the disadvantage of being a two-step process that can generate a mixture of products, as shown below:

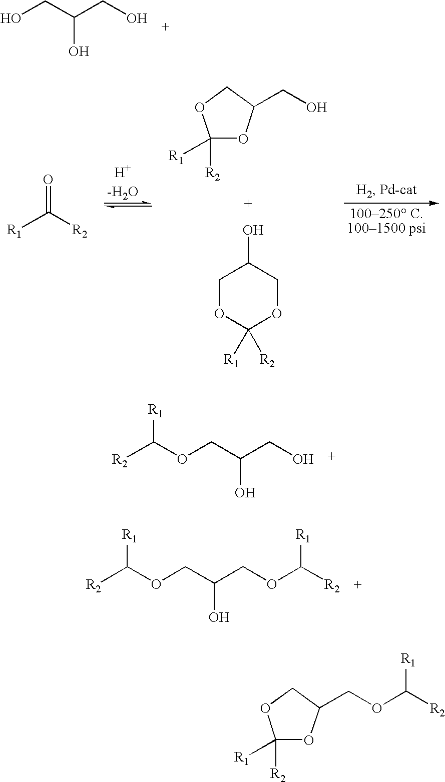

### Because this process can result in the production of a substantial amount of unwanted byproducts, it can be inefficient and thus, not desired.

Another concern common to all of the previously described processes is that none easily allow for the use of crude glycerol. Crude glycerol can be generated by a variety of processes and may contain a multitude of undesired impurities including water, inorganic salts such as chloride, sulfate, phosphate, acetate salts and others, organic compounds such as fatty acids, mono-glycerides and di-glycerides, phosphorolipids, protein residues, methanol, acids, bases and combinations thereof. The use of crude glycerol in reactions such as those previously described can be problematic since the impurities present in the crude glycerol can interfere with the reaction process or result in the formation of greater quantities of unwanted byproducts. One way to manage these impurities is to refine the crude glycerol prior to use to achieve at least about 99% purity, as is done in existing conventional processes. Unfortunately, while these purification processes can be effective, refining glycerol is an energy intensive process and can be extremely costly. Thus, refined glycerol can be considered to be a less than ideal feedstock for use in producing glycerol derivatives, such as alkyl glycerol ethers, because it can add substantial costs to the production process.

Therefore, there remains a need for a lower cost process for making alkyl glycerol ethers from crude glycerol that can prevent the formation of significant quantities of undesired by-products.

### SUMMARY OF THE INVENTION

Exemplary embodiments of the present invention generally relate to processes for converting glycerol to alkyl glycerol ethers.

In particular, in one exemplary embodiment, the processes generally relate to providing crude glycerol, alkyl alcohol and an etherification catalyst, providing an etherification reactor, and adding the crude glycerol, alkyl alcohol and etherification catalyst to the etherification reactor and reacting the crude glycerol, alkyl alcohol and etherification catalyst in the etherification reactor to obtain a reaction product comprising alkyl glycerol ethers.

In another exemplary embodiment, the processes generally relate to providing crude glycerol, alkyl alcohol and an etherification catalyst, providing an inert gas, providing an etherification reactor, providing a vaporizer, adding the crude glycerol and the inert gas to the vaporizer to obtain a glycerol vapor, transferring the glycerol vapor and inert gas to the etherification reactor, and adding the alkyl alcohol and etherification catalyst to the glycerol vapor in the etherification reactor in the presence of the inert gas and reacting the glycerol vapor, alkyl alcohol and etherification catalyst in the etherification reactor to obtain a vaporized reaction product comprising glycerol alkyl ethers.

In a further exemplary embodiment, the processes generally relate to providing crude glycerol, alkyl alcohol and an etherification catalyst, providing an etherification reactor, treating the crude glycerol to obtain a treated glycerol, adding the treated glycerol, alkyl alcohol and etherification catalyst to the etherification reactor and reacting the treated glycerol, alkyl alcohol and etherification catalyst in the etherification reactor to obtain a reaction product comprising at least one of mono-alkyl glycerol ethers, di-alkyl glycerol ethers, unreacted alkyl alcohol, unreacted glycerol, diglycerols, di-alkyl ethers, alkenes, water, etherification catalyst and combinations thereof, separating mono-alkyl glycerol ether from the di-alkyl glycerol ether, unreacted alkyl alcohol, unreacted glycerol, diglycerols, di-alkyl ethers, alkenes, water, and catalyst, and collecting the mono-alkyl glycerol ether.

### BRIEF DESCRIPTION OF THE DRAWINGS

FIG. 1 is a schematic flowchart representing an exemplary embodiment of a process in accordance with the present invention.

### DETAILED DESCRIPTION OF THE INVENTION

### A. Definitions

As used herein, “comprising” means the various components, ingredients, or steps, which can be conjointly employed in practicing the present invention. Accordingly, the term “comprising” encompasses the more restrictive terms “consisting essentially of” and “consisting of.”

As used herein, “glycerol” may refer to crude glycerol that may contain numerous impurities, including, but not limited to, water, inorganic salts such as chloride, sulfate, phosphate, acetate salts and others, organic compounds such as fatty acids, fatty esters, mono-glycerides, di-glycerides, phosphorolipids, protein residues, methanol, acids, bases and combinations thereof. Impurities may account for from about 10% to about 90% of the crude glycerol, and in one embodiment from about 10% to about 50% of the crude glycerol, by weight. Crude glycerol may be obtained in the course of the production of biodiesel, or from the conversion of fats/oils of plant or animal origin through saponification, trans-esterification or hydrolysis reactions. The crude glycerol may be liquid glycerol, or optionally, glycerol vapor. Optionally, as used herein, “glycerol” may also refer to “treated glycerol” as defined herein below.

As used herein, “reaction components” generally refers to glycerol, an alkyl alcohol and an etherification catalyst. In addition, “reaction components” may include an inert gas.

As used herein, “reaction product” means the composition(s) resulting from or remaining after reacting the (optionally treated) glycerol, alkyl alcohol and etherification catalyst in the etherification reactor. The reaction product may comprise alkyl glycerol ethers (specifically, mono-alkyl glycerol ethers and di-alkyl glycerol ethers), and any of unreacted glycerol, unreacted alkyl alcohol, diglycerols, di-alkyl ethers, alkenes, water, etherification catalyst and combinations thereof.

As used herein, “refined glycerol” means glycerol that is at least about 99% pure (i.e. containing less than about 1% impurities).

As used herein, “treated glycerol” means glycerol that has undergone a treating process such that the treated glycerol comprises greater than about 1% to about 10% impurities.

As used herein, “treating” means removing at least a portion of the impurities from the crude glycerol. “Treating” may be accomplished by a variety of methods, including, but not limited to neutralization, precipitation, filtration, evaporation, steam stripping, ion-exchange, adsorption and combinations thereof.

All percentages are by weight unless otherwise specified

### B. Processes for Making Alkyl Glycerol Ethers

Alkyl glycerol ethers are ether lipids that can be represented by the general formula:

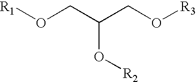

where R₁, R₂ and R₃ are independent of one another and may represent H, C₁₋₃₀ straight chained or branched, alkyl, alkylaryl or substituted or unsubstituted alkylphenyl. Embodiments of the processes set forth herein may be used to prepare such alkyl glycerol ethers via the reaction of glycerol, alkyl alcohol and a catalyst to obtain a reaction product comprising alkyl glycerol ethers.

Embodiments of the present processes can generally begin by providing “reaction components,” which may comprise glycerol, an alkyl alcohol, an etherification catalyst and, optionally, an inert gas, as set forth herein below.

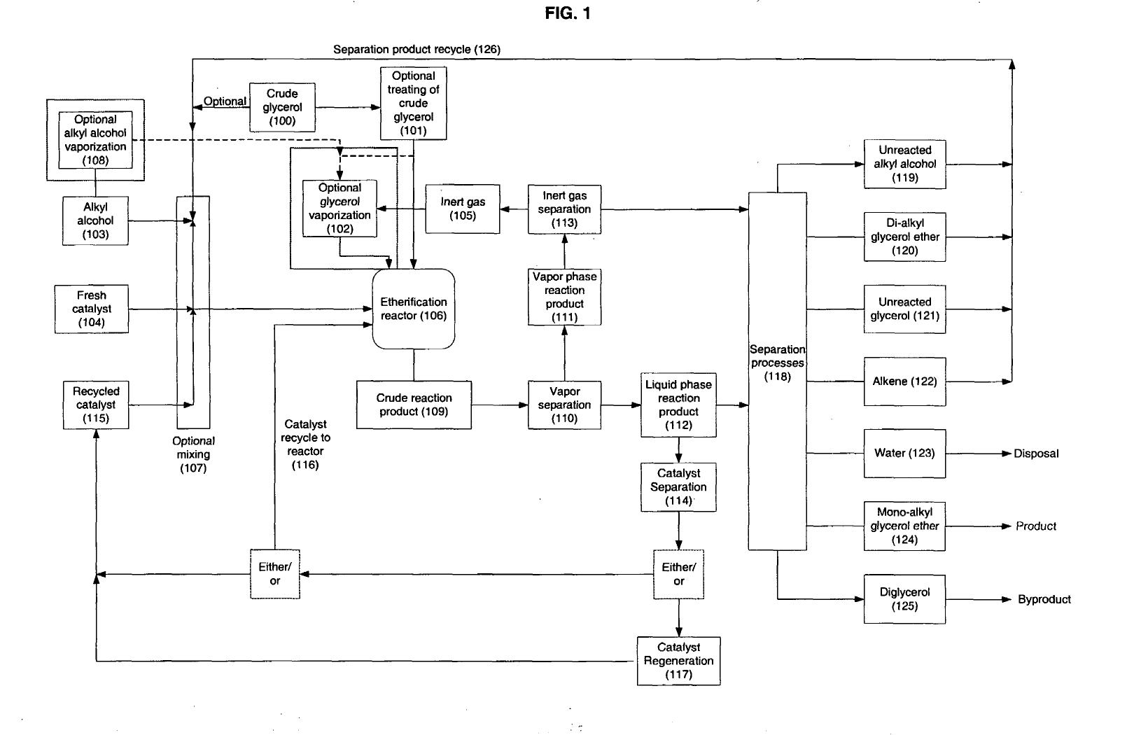

Referring to FIG. 1, embodiments of the present invention may initially comprise providing glycerol. (100). As used herein, “glycerol” may refer to crude glycerol that may contain numerous impurities, including, but not limited to, water, inorganic salts such as chloride, sulfate, phosphate, acetate salts and others, organic compounds such as fatty acids, fatty ester, mono-glycerides, di-glycerides, phosphorolipids, protein residues, methanol, acids, bases and combinations thereof. Impurities may account for at least about 10% of the crude glycerol, in one embodiment from about 10% to about 90% of the crude glycerol, and in yet another embodiment from about 10% to about 50% of the crude glycerol, by weight. Such crude glycerol may be obtained in the course of the production of biodiesel, or from the conversion of fats/oils of plant or animal origin through saponification, trans-esterification or hydrolysis reactions. As previously described, in most conventional processes, crude glycerol must first be refined prior to use in order to facilitate process control, maximize process yields, avoid catalyst poisoning and reduce impurities in the final reaction product. Because such refining can be costly, it is often not desirable. Embodiments described herein seek to address this issue by providing more cost-effective processes that allow for the use of crude glycerol without refinement.

Though the present embodiments will generally focus on the use of crude glycerol, they need not be limited to such. For example, in another embodiment, crude glycerol may be optionally treated prior to use in the processes described herein. Treating the crude glycerol (101) can aid in reducing the amount of impurities present without having to fully refine the crude glycerol. In this way, treating the crude glycerol can result in significant cost savings compared to refinement. As used herein, “treating,” the crude glycerol may be accomplished by a variety of methods, including, but not limited to neutralization, precipitation, filtration, evaporation, steam stripping, ion-exchange, adsorption and combinations thereof. Those skilled in the art will understand how the treatment of crude glycerol can be accomplished via the various methods set forth above, and that such treatment may vary depending on the impurities present.

Regardless of which treatment method is employed, the resulting “treated glycerol” may comprise from about 1% to about 10% of the aforementioned impurities by weight. This reduction in impurities in the treated glycerol can help provide better reaction yields of the desired alkyl glycerol ethers. Going forward, “glycerol” includes both crude and treated glycerol except where specifically designated as one or the other.

Alternately, rather than treating, the crude glycerol may be vaporized prior to further processing (102). As vapor phase reactions can be faster than liquid phase reactions, glycerol vapor may be desired such that the etherification reaction can be carried out in the vapor phase to speed up the reaction process. Glycerol vaporization may be carried out using any vaporizer known to those skilled in the art including, but not limited to, a flash tank evaporator or a wiped film evaporator. One skilled in the art would recognize that the conditions of temperature and pressure may vary according to the vaporization equipment used. An additional benefit of vaporizing the crude glycerol is that glycerol vaporization can reduce the amount of impurities present in the crude glycerol without having to fully refine the glycerol. In this way, using glycerol vapor can be a more cost effective option than using refined glycerol. However, it will be understood that the use of vaporization as a method of “treating” the crude glycerol, as described previously herein, may not be desirable as the use of vaporization independent of a vapor phase reaction can be energy intensive and thus, a less cost effective option.

Embodiments of the present processes may also comprise providing an alkyl alcohol (103). Alkyl alcohols can be generally represented by the formula:

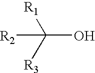

where, in one embodiment, R1 and R2 can independently represent a hydrogen atom or an alkyl group having from 1-29 carbon atoms while R3 can represent an alkyl group having from 1 to 29 carbon atoms. Some examples of alkyl groups having 1 to 29 carbon atoms include, but are not limited to, linear or branched, acyclic or cyclic, alkyl groups such as methyl, ethyl, n-propyl, i-propyl, n-butyl, i-butyl, sec-butyl, t-butyl, pentyl, cyclopentyl, hexyl, cyclohexyl, heptyl, octyl, 2-ethylhexyl, nonyl, decyl, undecyl, dodecyl, 4-butyloctyl, tridecyl, tetradecyl, pentadecyl, hexadecyl, heptadecyl, octadecyl, nonadecyl, eicosyl, isoheptadecyl, and isooctadecyl. In another embodiment, R2 and R3 can be taken together to form an alkylene group having from 2 to 21 carbon atoms. Some examples of alkylene groups having from 2 to 21 carbons include, but are not limited to, ethylene, trimethylene, tetramethylene, pentamethylene, and hexamethylene.

Such alcohols can be obtained by, for example, hydration of olefins, hydroformylation of an olefin with reduction of the resulting aldehyde, hydrogenation of fatty methyl esters, and direct hydrogenation of fatty acids. Those skilled in the art will understand that the choice of alcohol can vary depending on the target properties of the desired material. Similar to glycerol, the alkyl alcohol may be either in the liquid phase (103) or the vapor phase (108). The liquid phase may be desired when the alkyl alcohol boiling point is relatively high such as C₁₀-C₃₀ alcohols, while the vapor phase may be desired when the alkyl alcohol has a relatively low boiling point such as C₁-C₈ alcohols.

An etherification catalyst may also be provided. (104). Exemplary etherification catalysts for use herein may be any acid catalyst and may include, but are not limited to, Bronsted acids, Lewis acids or combinations thereof. The etherification catalyst may be a solid or a liquid. Those skilled in the art will understand how to select either a solid or liquid catalyst based on such factors as equipment and cost parameters. Some exemplary solid acid catalysts acceptable for use herein may include p-toluene sulfonic acid, naphthalenesulfonic acid, (±)-10-camphorsulphonic acid, xylenesulfonic acid, alkylbenzenesulfonic acid, kieselguhr impregnated with phosphoric acid, titania impregnated with phosphoric acid, heteropolyacid such as 12-tungstophosphoric acid or 12-molybdophosphoric acid supported in titania, clays or silica and/or alumina, cross-linked sulfonated polystyrene ion exchange resins such as Amberlyst™ (Rohm & Haas, USA, PA), polyperfluorosulfonic acid resin such as Nafion® (Dupont, USA, Delaware), with or without silica nanocomposite, alkyl sulfonic polysiloxane resin such as Deloxan ASP and natural and synthetic zeolites. Similarly, some exemplary liquid acid catalysts acceptable for use in the processes herein may include sulfuric acid, methanesulfonic acid, trifluoromethanesulfonic acid, linear alkyl sulphonic acid (HLAS), and combinations thereof. Further disclosure on suitable catalysts can be found in Appl. Chem., 2000, Vol. 72, No. 7, pp. 1313-1319 and Heteropoly Acids: Chem. Rev. 1998, 98, pp. 171-198.

Optionally, an inert gas may also be provided (105) to help prevent oxidation reactions from taking place and facilitate the evaporation of glycerol. While any inert gas known to those skilled in the art may be acceptable for use herein, examples of inert gasses that may be useful in embodiments of the present processes can include the noble gases (e.g. helium or argon), nitrogen, carbon dioxide, superheated steam and combinations thereof. In one embodiment, the inert gas may comprise nitrogen.

Once the glycerol, alkyl alcohol and etherification catalyst are provided, they may be added to an etherification reactor (106), optionally in the presence of the inert gas (105), to initiate the reaction. While the molar ratio of glycerol to alkyl alcohol may vary, in one embodiment, the molar ratio may be from about 1:30 to about 1:0.1, and in another embodiment from about 1:5 to about 1:0.5. Additionally, while the ratio of catalyst to glycerol may also vary, in one embodiment, it may be from about 0.001:1 to about 0.1:1 by weight. One skilled in the art will recognize that the amount of catalyst used can vary depending on such factors as the strength of the catalyst, and in the case of solid catalysts, the amount of surface area available per gram of catalyst. The amount of catalyst used may also be varied to alter the speed at which the reaction takes place. For instance, faster reactions can be advantageous because they generally allow for the use of more compact reaction equipment and can result in the formation of fewer byproducts, while slower reactions can be advantageous because they can often be carried out using less catalyst, which can lead to lower operating costs.

Any etherification reactor known to those skilled in the art may be used herein and may include a fixed bed column reactor such as a trickle bed reactor, a continuous stirred tank reactor, a reactive distillation reactor, a batch reactor, or combinations thereof. It will be understood that the manner in which the glycerol, alkyl alcohol and etherification catalyst are fed into the etherification reactor can vary depending on the equipment used and the phase of each reaction component. For instance, as shown in FIG. 1, when using liquid glycerol, the glycerol may optionally be premixed with the other reaction components (107) prior to the addition of the mixture into the etherification reactor or the glycerol may be added to the reactor independently of the remaining reaction components (100). Similarly, if glycerol vapor is used, it may be added alone (102) or in combination with vaporized alkyl alcohol (108). In this situation, one skilled in the art will understand that it may be more advantageous to have the catalyst already in place in the reactor prior to the addition of the glycerol vapor and alkyl alcohol vapor since it may simplify the process of contacting the vaporized reaction components with the catalyst.

Regardless of the manner of introduction of the various reaction components, once inside the etherification reactor, the glycerol, alkyl alcohol and catalyst may react, in the presence of the inert gas if included, to produce a reaction product comprising alkyl glycerol ethers (109). While not intending to be limited by theory, it is believed that the acid catalyst can convert the hydroxyl group of the alkyl alcohol into a better leaving group through the formation of an oxonium ion. The alkyl glycerol ether can then be formed by the nucleophilic attack of the glycerol on the oxonium ion in either a bimolecular reaction, S_{N}2 (primary and secondary alcohols), or a reaction with the olefin generated from the dehydration of the alcohol (secondary, tertiary, allylic, aryl alcohols). Alternately, the primary hydroxyl group of glycerol may react with the acid catalyst to form the oxonium ion, which can then react with the alkyl alcohol.

In view of the above, it will be understood that reaction conditions can vary depending on the particular reaction components (i.e. glycerol, alkyl alcohol, catalyst) selected. However, in one embodiment the reaction between the glycerol, alkyl alcohol and etherification catalyst may occur in the etherification reactor at a temperature of from about 70° C. to about 250° C., and in one embodiment from about 110° C. to about 180° C., and in still another embodiment from about 120° C. to about 150° C., and at a pressure of from about 0.025 atm to about 50 atm. The reaction may be permitted to continue until at least about 30% of the glycerol has been converted to alkyl glycerol ether, in one embodiment at least about 50%, in another embodiment at least about 70% and in yet another embodiment at least about 90% of the glycerol has been converted to alkyl glycerol ether. Likewise, the time needed to carry out the reaction can vary depending on the reaction components used, however, in one embodiment, the reaction can be carried out for from about 1 minute to about 600 minutes. Those skilled in the art will understand how to select the proper process parameters based on the reaction components and equipment used, as well as the reaction product desired.

As used herein, “reaction product” means the composition(s) resulting from or remaining after the reacting the (optionally treated) glycerol, alkyl alcohol and etherification catalyst in the etherification reactor, which may comprise alkyl glycerol ethers (specifically, mono-alkyl glycerol ethers and di-alkyl glycerol ethers), and any of unreacted glycerol, unreacted alkyl alcohol, diglycerols, di-alkyl ethers, alkenes, water, etherification catalyst and combinations thereof. The state of the reaction product may be a liquid reaction product (“liquid phase”), a vaporized reaction product (“vapor phase”), or a combination thereof. It will be understood by those skilled in the art that the phase of each reaction product can vary depending on the reaction components used.

Once the reaction product is obtained, further separation may be carried out if so desired. For instance, initially, the reaction product may be separated into a vapor phase reaction product (111) and a liquid phase reaction product (112) to make the subsequent processing steps easier as it is generally easier to handle and move single phase streams than multi-phase streams. This separation may occur either while the reaction product is still in the etherification reactor, or after the reaction product has been removed from the etherification reactor (110), as shown in FIG. 1.

When handling the vapor phase reaction product, the inert gas (if used) may be separated from the remaining vapor phase reaction products (113) and recycled back to the etherification reactor for reuse (106).

Similarly, when handling the liquid phase reaction product, the catalyst may be separated (114) from the remaining liquid phase reaction products (112) to help prevent unwanted reactions from occurring during subsequent processing of the liquid reaction product. The catalyst may be recycled (115) back to one of the streams feeding the reaction components to the etherification reactor (106), or it may optionally be recycled directly back into to the etherification reactor (116). Since the catalyst may lose some of its activity over time, it may be desirable to regenerate the catalyst activity (117) prior to returning the catalyst to the reaction components (115) or etherification reactor.

The remaining reaction products may be further separated from one another in one or more separation processes (118) using any appropriate method known to those skilled in the art so as to obtain individual products (i.e. unreacted alkyl alcohol (119), di-alkyl glycerol ether (120), unreacted glycerol (121), alkene (122), water (123), mono-alkyl glycerol ether (124) and diglycerol (125). As will be understood by those skilled in the art, the specific separation processes used and the degree of separation of the reaction products may depend on the specific alcohols used in the etherification reaction and the desired purity of the reaction products. For example, it may be desirable to recycle (126) the unreacted alkyl alcohol (119) and unreacted glycerol (121) for reuse to save on raw material costs. Additionally, it may desirable to concurrently recycle (126) any di-alkyl glycerol ether (120) and/or alkenes (122) since these by-products can skew the reaction equilibrium toward the desired mono-alkyl glycerol ethers, thereby enhancing the production thereof. Water (123) and diglycerol (125) may be considered byproducts of the reaction and, thus, can be separated and removed from the other reaction products and either processed for further use in another application, or disposed. Finally, the mono-alkyl glycerol ether (124) may be collected as the desired product. The following representative embodiments of such separation processes are included for purposes of illustration and not limitation.

More specifically, for example, if reacting methanol with glycerol to form a reaction product comprising glycerol methyl ethers, at the end of the reaction, water, dimethyl ether and methanol may be in the vapor phase at reaction temperatures while the alkyl glycerol ether, di-alkyl glycerol ether, and diglycerol may be in the liquid phase. The vapor phase reaction products may be conveniently vented off from the liquid phase reaction products and then further separated into individual components for reuse if appropriate and/or desired. Since the boiling points of these vapor phase reaction products are significantly different, they could be separated, for example, via a series of condensers that condense one vaporized product a time. The water (byproduct) can be disposed and the liquid methanol may be recycled to the etherification reactor. Dimethyl ether (boiling point of about −22° C.) may be recycled to the etherification reactor as a vapor or condensed and recycled as a liquid. Similarly the liquid phase reaction products, including the alkyl glycerol ether, di-alkyl glycerol ether, and diglycerol could be separated by taking advantage of their differing boiling points and using one or more distillation columns to obtain the individual components.

As another example, when reacting isopropanol with glycerol to form isopropyl glycerol ethers, the propene, water, isopropanol and di-isopropyl ether formed during the reaction may be vapor phase reaction products while the isopropyl glycerol ethers, di-isopropyl glycerol ethers, and diglycerols also formed during the reaction may be in the liquid phase. The vapor phase reaction products can conveniently be removed from the etherification reactor by way of, for example, a flash tank, while the liquid phase reaction products, including the isopropyl glycerol ethers, can remain in the etherification reactor for subsequent separation and collection.

As yet another example, when reacting octadecyl alcohol with glycerol water may be in the vapor phase at reaction temperatures while the remaining reaction products may be in the liquid phase. The vaporized water may be conveniently vented off during or after the reaction. The remaining liquid reaction products, which may be solids at lower temperatures, can be cooled and separated using a method such as, for example, crystallization or solvent extraction.

After further separation is complete, the alkyl glycerol ethers, and specifically, the mono-alkyl glycerol ethers, obtained from embodiments described herein may be used in a wide variety of products as, for example, antimicrobials, emulsifiers, surfactants, fragrance enhancers, moisture retaining agents, solvents, and solvatropes.

In view of the above, it will be understood that embodiments of the present processes may be carried out using either batch or continuous mode.

### EXAMPLES

### Example 1

About 140 grams of 2-propanol is added to a 500 ml Parr® (Parr Instrument Company, USA, IL) model 4843 pressure etherification reactor along with about 215 grams of glycerol and about 46.7 grams of Amberyst 35W™ (Rohm & Haas, USA, PA) catalyst. The top is placed on the etherification reactor and bolted down. Extending into the etherification reactor from the top is an agitator shaft with turbine blades attached at two places, at the bottom of the shaft and half-way up the shaft. Also extending into the etherification reactor from the top are a liquid sampling line with a sintered metal filter at the end of the sampling line, a thermocouple, and a vapor space vent line. The reaction components in the reactor are heated to a temperature of about 150° C. and a pressure of between about 270 psi to 320 psi for about 6 hours with an agitator speed of about 425 rpm. After about 6 hours the reactor is cooled to about room temperature and the reaction product and catalyst are collected in a beaker. This material is filtered using a Buchner-type filter funnel, Whatmann 40 filter paper, and an Erlenmeyer flask connected to a sink aspirator. The filtered reaction products in the Erlenmeyer flask are transferred to a 500 ml round bottom flask. The flask is placed on a Labconco evaporator and the water, di-isopropyl ether, unreacted 2-propanol, and propene are removed under vacuum. The remaining liquid reaction product consists of about 43% glycerol mono-isopropyl ether, about 14% glycerol di-isopropyl ether, and about 43% unreacted glycerol, all by weight.

### Example 2

About 134 grams of methanol is added to a 500 ml Parr® (Parr Instrument Company, USA, IL) model 4843 etherification reactor, as described in Example 1, along with about 194 grams of glycerol and about 19.2 grams of Amberyst 15™ (Rohm & Haas, USA, PA) catalyst. The top is placed on the etherification reactor bolted down. The reaction components in the reactor are heated to a temperature of about 150° C. and a pressure of between about 165 psi to 425 psi for about 4 hours with an agitator speed of about 425 rpm. At the end of about 4 hours the reactor is cooled to about room temperature and the reaction product and catalyst are collected in a beaker. This material is filtered using a Buchner-type filter funnel, Whatmann 40 filter paper, and an Erlenmeyer flask connected to a sink aspirator. The filtered reaction products in the Erlenmeyer flask are transferred to a 500 ml round bottom flask. The flask is placed on a Labconco evaporator and the water, dimethyl ether, and unreacted methanol, are removed under vacuum. The remaining liquid reaction product consists of about 46% glycerol mono-methyl ether, about 7% glycerol di-methyl ether, about 47% unreacted glycerol, and less than 1% diglycerol ether, all by weight.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A process for converting glycerol to alkyl glycerol ethers comprising:
a) providing glycerol, alkyl alcohol and an etherification catalyst;
b) providing an etherification reactor;
c) adding the glycerol, alkyl alcohol and etherification catalyst to the etherification reactor; and
d) reacting the glycerol, alkyl alcohol and etherification catalyst in the etherification reactor to obtain a reaction product comprising alkyl glycerol ethers.

2. The process of claim 1 wherein the glycerol and alkyl alcohol are added to the etherification reactor in molar ratio of glycerol to alkyl alcohol of from about 1:30 to about 1:0.5.

3. The process of claim 1 wherein reacting the glycerol, alkyl alcohol and etherification catalyst occurs at a temperature of from about 70° C. to about 250° C.

4. The process of claim 1 wherein the reaction product comprises a liquid reaction product comprising alkyl glycerol ethers and a vapor reaction product comprising volatile components.

5. The process of claim 4 comprising separating the liquid reaction product comprising alkyl glycerol ethers from the vapor reaction product.

6. The process of claim 1 further comprising:
providing an inert gas; and
adding the glycerol, alkyl alcohol and etherification catalyst to the etherification reactor and reacting the glycerol, alkyl alcohol and etherification catalyst in the etherification reactor in the presence of the inert gas to obtain a liquid reaction product comprising alkyl glycerol ethers and a vapor reaction product comprising volatile components and the inert gas.

7. The process of claim 6 further comprising removing the volatile components from the inert gas.

8. The process of claim 7 further comprising recycling the inert gas.

9. The process of claim 1 wherein the etherification reactor is a fixed bed column reactor, a continuous stir tank reactor, a reactive distillation reactor or a batch reactor.

10. The process of claim 6 wherein the inert gas comprises nitrogen.

11. The process of claim 1 wherein the etherification catalyst is selected from the group consisting of Bronsted acids, Lewis acids and combinations thereof.

12. The process of claim 1 further comprising treating the glycerol to obtain a treated glycerol and adding the treated glycerol, alkyl alcohol and etherification catalyst to the etherification reactor.

13. The process of claim 12 wherein treating the glycerol comprises a method selected from the group consisting of neutralization, precipitation, filtration, evaporation, steam stripping, ion-exchange, adsorption and combinations thereof.

14. The process of claim 2 wherein the molar ratio of glycerol to alkyl alcohol of from about 1:5 to about 1:1.

15. A process for converting glycerol to alkyl glycerol ethers comprising:
a) providing crude glycerol, alkyl alcohol and an etherification catalyst;
b) providing an inert gas;
c) providing an etherification reactor;
d) providing a vaporizer;
e) adding the crude glycerol and the inert gas to the vaporizer to obtain a glycerol vapor;
f) transferring the glycerol vapor and inert gas to the etherification reactor; and
g) adding the alkyl alcohol and etherification catalyst to the glycerol vapor in the etherification reactor in the presence of the inert gas; and
h) reacting the glycerol vapor, alkyl alcohol and etherification catalyst in the etherification reactor to obtain a vapor reaction product comprising glycerol alkyl ethers.

16. The process of claim 15 wherein the reacting the glycerol vapor, alkyl alcohol and etherification catalyst occurs at a temperature of from about 70° C. to about 250° C.

17. The process of claim 15 wherein the glycerol and alkyl alcohol are added to the etherification reactor in molar ratio of glycerol to alkyl alcohol of from about 1:30 to about 1:0.5.

18. The process of claim 15 further comprising recycling unreacted glycerol to the evaporator.

19. The process of claim 15 wherein the vaporizer is a flash tank evaporator or a wiped film evaporator.

20. A process for converting glycerol to alkyl glycerol ethers comprising:
a) providing crude glycerol, alkyl alcohol and an etherification catalyst;
b) providing an etherification reactor;
c) treating the crude glycerol to obtain a treated glycerol;
d) adding the treated glycerol, alkyl alcohol and etherification catalyst to the etherification reactor;
e) reacting the treated glycerol, alkyl alcohol and etherification catalyst in the etherification reactor to obtain a reaction product comprising at least one of mono-alkyl glycerol ethers, di-alkyl glycerol ethers, unreacted alkyl alcohol, unreacted glycerol, diglycerols, di-alkyl ethers, alkenes, water etherification catalyst and combinations thereof;
f) separating mono-alkyl glycerol ether from the di-alkyl glycerol ether, unreacted alkyl alcohol, unreacted glycerol, diglycerols, di-alkyl ethers, alkenes, water, and etherification catalyst; and collecting the mono-alkyl glycerol ether.
